(19) United States
(12) Patent Application Publication
(10) Pub. No.: US 2010/0137858 A1
(43) Pub. Date: Jun. 3, 2010

(54) Coil Guide

(75) Inventors: Robert L. Rykhus, JR., Edina, MN (US); Vincent G. Copa, Minnetonka, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/628,571

(22) Filed: Dec. 1, 2009

Related U.S. Application Data
(60) Provisional application No. 61/118,810, filed on Dec. 1, 2008.

(51) Int. Cl. A61B 18/18

(52) U.S. Cl. 606/33

(57) ABSTRACT

A catheter including a coil guide that assist with the proper installation of an antenna for the delivery of microwave energy on a catheter. The coil guide secures the antenna in the proper position and shape while the catheter is navigated to the targeted tissue. The coil guide includes a channel that is molded or cut into the coil guide for receiving the antenna and maintaining proper position of the antenna. The channel provides a guide for medical personnel when wrapping the antenna coils onto the catheter body and secures the antenna in the proper shape and position while the catheter is being navigated to the targeted tissue. Alternatively, the antenna can be pre-coiled for installation into the channel. The coil guide can be adhesively attached or overmolded proximate a distal end of a catheter body.

### PRIORITY CLAIM

The present application claims priority to U.S. Provisional Application Ser. No. 61/118,810, filed Dec. 1, 2008, and entitled “COIL GUIDE”, which is hereby incorporated by reference in its entirety.

### FIELD OF THE DISCLOSURE

The present invention relates to the thermotherapy treatment of biological tissue, particularly prostatic tissue, through the delivery of microwave energy to targeted tissue via a catheter having a microwave antenna. More specifically, the present invention is directed at a coil guide for assisting medical personnel in the installation of microwave antennas on a catheter body and securing the microwave antenna in place while the catheter is positioned proximate to the targeted tissue.

BACKGROUND OF THE DISCLOSURE A commonly employed medical treatment for a variety of ailments is the direct heating or irradiation of affected biological tissue. For example, Benign Prostatic Hyperplasia (BPH), or an enlarged prostate, is commonly treated by TransUrethral Microwave Thermotherapy (TUMT). TUMT treats BPH by applying directed microwave energy at specific portions of the prostate to destroy targeted portions of the prostatic tissue that are causing the condition. TUMT, as with most direct heating or irradiating treatments, carefully directs heat or microwave energy at specific tissue to destroy only the targeted portions of the biological tissue and minimize damage to the surrounding tissue. However, one of the primary challenges associated with this type of treatment is heating or irradiating only the targeted tissue. Another primary challenge associated with this type of treatment is applying the correct amount of heat or radiation to the targeted tissue. The shape and relative position on the catheter body of the antenna generating the heat or radiation can influence whether the correct tissue is treated or whether the appropriate amount of heat or radiation is applied to the targeted tissue.

Heat or microwave energy is typically applied to targeted tissues by navigating a catheter having a heating or microwave antenna through a biological lumen, such as, for example, the urethra, to the tissue to be treated. Once the catheter has been positioned within the lumen, the antenna is in contact with the targeted tissue. The catheters and antennas typically employed in such procedures often resemble the Rx-200™ catheter for use with the TMX3000™ Office Thermo Therapy™ System, all of which are produced by American Medical Systems, of Minnetonka, Minn. The Rx-200™ and other similar catheters employ a helical shaped antenna that is coiled around the catheter at or near the tip of the catheter. The antenna may be attached to the catheter pre-coiled or wrapped onto the catheter in the coiled shape. The later approach can be advantageous as it allows medical personnel to define the exact length of the heating or irradiating antenna and tailor the length of the antenna to the dimensions or shape of the targeted tissue. However, medical personnel must be particularly skilled to effectively wrap the antenna because the antenna must be wrapped such that the antenna coils are evenly spaced. If the coils are not evenly spaced, the coils may be bunched too closely whereby the radiation is concentrated in too small an area. Alternative, if the coils are stretched too far apart, the radiation can be diffused over too great an area. Even if the antenna is pre-coiled the coils may still become bunched or stretched when the antenna is attached to the catheter.

A related issue to properly spacing the antenna coils during installation is maintaining the proper spacing of the antenna coils while the catheter is maneuvered through the patient's body to the targeted tissue. Once inserted into the patient's body, the catheter must be navigated through the biological lumen of the patient's body to the targeted tissue. Upon reaching the general area where the targeted tissue is located, the relative position of the catheter may have to be adjusted within the biological lumen to accurately position the antenna proximate to the targeted tissue. The bending or flexing of the catheter or frictional forces from the antenna coils brushing against the walls of the biological lumen may cause the antenna coils to become bunched up or stretched apart.

Once the antenna is attached to the catheter and the catheter is inserted into the patient's body, the position of the antenna relative to the targeted tissue can only be adjusted by shifting the relative position of the catheter within the biological lumen. As such, the antenna must be placed at a specific position along the length of the catheter such that medical personnel adjusting only the position of the catheter can accurately position the antenna proximate to the target tissue. However, as the antenna and the catheter are often small in size to navigate the narrow biological lumen, positioning the antenna on a specific point on the catheter may be difficult to perform without assistance.

### SUMMARY OF THE DISCLOSURE

The present disclosure provides a coil guide and related methods of using said coil guide for assisting medical personnel in the installation of an antenna on the catheter and securing the antenna in the proper position and shape while the catheter is navigated to the targeted tissue. More specifically, the present disclosure teaches the use of a coil guide comprising a continuous channel that defines the proper position of the antenna. The continuous channel provides a guide to follow when wrapping antenna coils onto the catheter body. The continuous channel secures the antenna in the proper shape and position on the catheter body while the catheter is being navigated to the targeted tissue. The coil guide assists in properly installing an antenna on the catheter without causing bunching or stretching of the antenna coils. Not only does the coil guide assist in installing antennas with properly spaced coils, but the coil guide also maintains proper coil spacing while the catheter is navigated through the patient's body to the targeted tissue and when any subsequent adjustments are made to align the antenna with the targeted tissue. The present invention also indicates where on a catheter the antenna should be properly attached and assists in placing the antenna at that position.

In one aspect, the present disclosure is directed at a coil guide for assisting in attaching a pre-coiled antenna to a catheter or wrapping an antenna on to a catheter and holding the antenna in the correct place and shape while the catheter is navigated to the targeted tissue. Generally, the coil guide can comprise a continuous channel formed therein, wherein the channel defines the proper position and shape for the antenna. The continuous channel can be cut into a perimeter surface of the coil guide or alternatively, the continuous channel can be molded into the coil guide during fabrication of the coil guide.

In another aspect, the present disclosure provides a method of attaching an antenna to a catheter and fixing the antenna in place while the catheter is navigated to the targeted tissue or adjusted to properly align the antenna proximate to the targeted tissue. The method can comprise a step of providing a coil guide having a continuous channel that defines the proper shape, spacing and position for the antenna relative to a distal treatment end of the catheter. In some embodiments, the continuous channel is molded into the coil guide during initial fabrication. In some embodiments, the continuous channel can be cut into the coil guide either before or after the coil guide is attached to the catheter. The method can further comprise the step of fixedly attaching the coil guide to the catheter using a suitable attachment process such as, for example, overmolding, adhesive bonding or sonic welding. The method can also comprise the step of positing the antenna within the continuous channel to define a helical antenna. In some embodiments, a continuous wire can be wrapped around the coil guide wherein each antenna coil resides within the continuous channel. Alternatively, the continuous wire can be pre-coiled into a helical antenna that is positioned over the coil guide and essentially snaps into position within the continuous channel.

The above summary of the invention is not intended to describe each illustrated embodiment or every implementation of the present invention. The Figures and the detailed description that follow more particularly exemplify these embodiments.

### BRIEF DESCRIPTION OF THE FIGURES

The invention can be more completely understood in consideration of the following detailed description of various embodiments of the invention in connection with the accompanying drawings, in which:

FIG. 1 is a side view of a prior art treatment catheter body having an antenna wrapped about the catheter body.

FIG. 2 is an exploded side view of a catheter body having a coil guide according to a representative embodiment of the present invention.

FIG. 3 is a side view of an embodiment of a coil guide according to a representative embodiment of the present invention.

FIG. 4 is a partial side view of a treatment catheter according to a representative embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE FIGURES

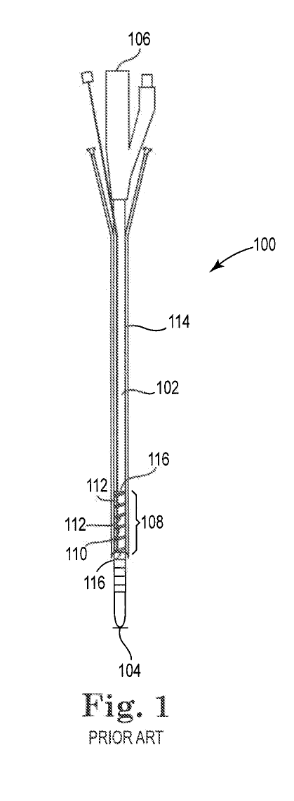

Referring now to FIG. 1, a representative treatment catheter 100 of the prior art generally comprises an elongated, tubular catheter body 102 having a distal treatment end 104 and a proximal handling end 106. Treatment catheter 102 further includes a helical antenna 108 wrapped about the catheter body 102. The helical antenna 108 generally comprises a continuous wire 110 wrapped about the catheter body 102 such that a plurality of wire coils 112 form the helical appearance of the helical antenna 108. Catheter body 102 generally comprises a smooth exterior surface 114 that is incapable of guiding the continuous wire 110 into the appropriate position about the catheter body 102 as well as holding the wire coils 112 in alignment after the helical antenna 108 is installed. As such, the wire coils 112 are often improperly wrapped or attached causing the antenna coils 112 to be unevenly spaced. In the event that the antenna coils 112 are unevenly spaced, the treatment catheter 100 can suffer from uneven heating or irradiating of targeted tissue. Similarly, end wire coils 116 at the edges of the helical antenna 108 can become stretched away from their adjacent wire coils 112 or alternatively, the end wire coils 116 can become bunched together with the adjacent wire coils 112 resulting in the effective area heated or irradiated by the antenna to change substantially. If the effective area of the helical antenna 108 is shrunk or expanded, the amount of energy desired to be imparted to the treatment area will be affected so as to impact the overall success of the treatment. Depending on how the shape of the helical antenna 108 is affected, the effective treatment area of the treatment catheter 100 may not treat enough of the targeted tissue if the effective area is shrunk or can result in the treatment of untargeted, healthy tissue if the effective area is increased.

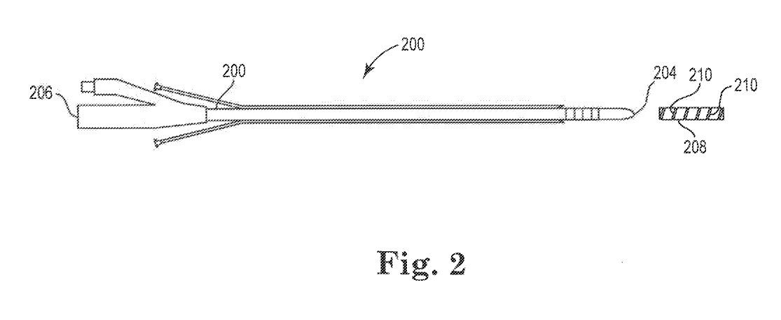

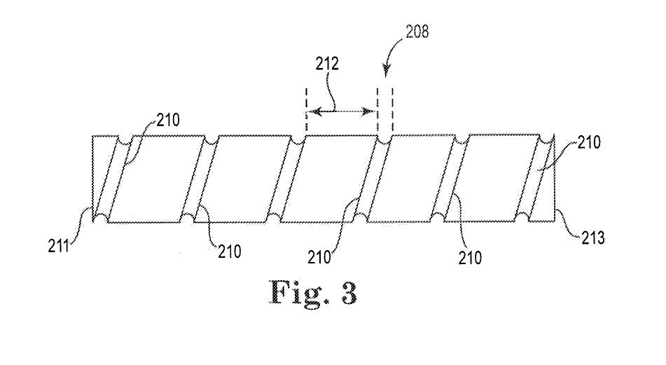

In contrast, a representative embodiment of a treatment catheter 200 of the present invention generally comprises a catheter body 202 having a distal treatment end 204 and a proximal handling end 206 as illustrated in FIG. 2. The treatment catheter 200 further comprises a tubular coil guide 208 having a continuous channel 210 formed therein as shown in FIG. 3. The continuous channel 210 is continuous between a first end 211 and a second end 213 of the coil guide 208. As the continuous channel 210 wraps about the coil guide 208, adjacent portions of the continuous channel 210 are separated by a channel distance 212.

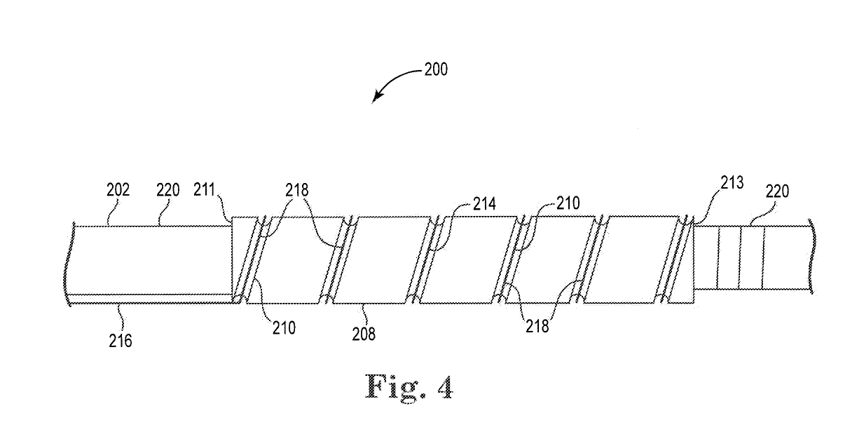

Referring to FIG. 4, the coil guide 208 is generally attached to catheter body 202 by slidably advancing the coil guide 208 over the distal treatment end 204 such that the coil guide resides proximate the distal treatment end 204. Once the coil guide 208 is positioned along the catheter body 202, the coil guide is fixedly attached to the catheter body 202. In a one representative embodiment, the coil guide 208 is overmolded onto the catheter body 202. Alternatively, the coil guide 208 can be attached through other suitable techniques including, for example, adhesive bonding or sonic welding.

With the coil guide 208 fixedly attached to the catheter body 202, a helical antenna 214 can be installed as illustrated in FIG. 4. Generally, the helical antenna 214 is comprised of a continuous wire 216 that is positioned within the continuous channel 210. By positioning the continuous wire 216 within the continuous channel 210, adjacent coils 218 remain consistently space apart by channel distance 212 along the length of the continuous channel 210. With the helical antenna 214 positioned within the continuous channel 210, the coils 218 are prevented from sliding along a perimeter surface 220 of the catheter body 210. In addition, continuous channel 210 prevents the coils 218 from shifting relative to each other, thus maintaining spacing essentially equivalent to the channel distance 212. The continuous wire 216 can be wrapped onto the catheter guide 208 using the continuous channel 210 as the guide for forming the helical antenna 214. Alternatively, the continuous wire 216 can be pre-coiled and slid over the catheter guide 208 such that the antenna coils 218 align with the continuous channel 210. The continuous channel 210 has sufficient dept to retain the helical antenna 214 in the proper position on the catheter body 202 and maintain channel distance 212 between adjacent coils 218. The continuous 210 can be molded into the channel guide 208 or alternatively, the continuous channel 210 can be cut into the channel guide 208. In some embodiments, the continuous channel 210 can be cut into the channel guide 208 following attachment of the channel guide 208 to the catheter body 202. The helical antenna 214 can extend the entire length of the continuous channel 210 or can be occupy only a portion of the continuous channel 210 as dictated by the treatment procedure. The helical antenna 214 is generally capable of providing heat to the targeted tissue or irradiating the tissue with various forms of electromagnetic radiation, such as microwaves.

In another embodiment, the present disclosure provides for a method of retaining a helical antenna 214 on a catheter body 202 while maintaining the proper spacing between the adjacent coils 218. The method comprises a step of providing coil guide 208 and affixing the coil guide 208 proximate the distal treatment end of the catheter body 202. The coil guide 208 can be affixed to the catheter body 202 using an overmolding procedure or other suitable attachment methods including, for example, adhesive joining or sonic welding. The method can further comprise forming the continuous channel 210 in the coil guide. In some embodiments, the continuous channel 210 can be molded as part of the original fabrication of the coil guide 208, or alternatively, the continuous channel 210 can be cut into the coil guide 208. In some embodiments, the continuous channel 210 can be cut into the coil guide 208 following attachment of the coil guide 208 to the catheter body 202. Finally, the helical antenna 214 is mounted within the continuous channel 210 whereby channel distance 212 is maintained between adjacent coils 218. In some embodiments, continuous wire 216 can be wrapped about the coil guide 208 such that the continuous wire 216 resides within the continuous channel 210. Alternatively, the continuous wire 216 can be pre-coiled such that the pre-coiled portion can be positioned within the continuous channel 210.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

What is claimed:

1. A catheter having a fixed position coil, comprising:
a catheter body having a distal end;
a coil guide positioned proximal the distal end, the coil guide including a continuous channel formed within the coil guide, the continuous channel spiraling about the coil guide between a first end and a second end of the coil guide; and
an antenna coil positioned within the channel, wherein the orientation of the antenna coil about the catheter body is fixed by the coil guide.

2. The catheter of claim 1, wherein the coil guide is adhesively attached over the catheter body.

3. The catheter of claim 1, wherein the coil guide is overmolded over the catheter body.

4. The catheter of claim 1, wherein the antenna coil is wrapped into the continuous channel to position the antenna coil

5. The catheter of claim 1, wherein the antenna coil is pre-coiled and is slidably directed over the distal end and the coil guide to position the antenna coil into the continuous channel.

6. The catheter of claim 1, wherein the continuous channel is molded directly into the coil guide.

7. The catheter of claim 1, wherein the continuous channel is cut into the coil guide.

8. A method for fixing a coil antenna position on a catheter, comprising;
providing a coil guide having a continuous channel spiraling about the coil guide;
sliding the coil guide over a distal end of a catheter body such that the coil guide resides proximate the distal end;
attaching the coil guide to the catheter body; and
positioning an antenna coil within the continuous channel.

9. The method of claim 8, wherein the step of positioning the antenna coil within the continuous channel further comprises the steps of:
coiling the antenna coil to form a pre-coiled antenna coil; and
sliding the pre-coiled antenna coil over the distal end of the catheter body such that the pre-coiled antenna coil is mounted within the continuous channel.

10. The method of claim 8, wherein the step of positioning the antenna coil within the continuous channel further comprises the step of:
wrapping the antenna coil around the coil guide and into the continuous channel.

11. The method of claim 8, wherein the step of attaching the coil guide to the catheter body, further comprises the step of:
overmolding the coil guide to the catheter body.

12. The method of claim 8, wherein the step of attaching the coil guide to the catheter body, further comprises the step of:
coupling the coil guide to the catheter body with an adhesive.

13. The method of claim 8, wherein the step of providing the coil guide having the continuous channel spiraling about the coil guide, further comprises the step of:
molding the continuous channel into the coil guide.

14. The method of claim 8, wherein the step of providing the coil guide having the continuous channel spiraling about the coil guide, further comprises the step of:
cutting the continuous channel into the coil guide.
